(19) United States
(12) Patent Application Publication
(10) Pub. No.: US 2014/0336610 A1
(43) Pub. Date: Nov. 13, 2014

(54) METHODS AND DEVICES FOR POINT OF USE MIXING OF PHARMACEUTICAL FORMULATIONS

(71) Applicant: Biodel, Inc., Danbury, CT (US)

(72) Inventors: Gerard Michel, Ridgewood, NJ (US); Robert Feldstein, Yonkers, NY (US); Bryan Wilson, Brewster, NY (US); Jake Ganem, Danbury, CT (US)

(21) Appl. No.: 14/249,348

(22) Filed: Apr. 9, 2014

Related U.S. Application Data
(60) Provisional application No. 61/810,111, filed on Apr. 9, 2013.

(51) Int. Cl. A61J 1/22; A61M 5/14; B65D 81/32; B01F 3/08; B01F 5/04; B01F 15/02; A61K 38/28; A61M 5/315

(52) U.S. Cl. CPC A61J 1/22; A61K 38/28; A61M 5/1408; A61M 5/31596; B01F 3/0865; B01F 5/0405; B01F 15/0216; B65D 81/32; B01F 2215/0032
USPC 604/500; 514/5.9; 604/83; 366/152.2; 206/219

(57) ABSTRACT

Methods and devices for delivering variable amounts of a drug formulation directly from a drug delivery device without requiring point of use reconstitution as a separate mixing step are described herein. These methods and devices increase the convenience, speed and simplicity of administration of drug formulations. The devices mix two or more components in a constant mixing ratio effectively simultaneous to administration, and allow for variable doses while retaining the desired ratio of the components in each dose. The devices are particularly useful for administering drug formulations that are unstable at room temperature in liquid form. The methods described herein generally use conventional devices that have been modified to administer an unstable compound and provide acceptable storage stability. Typical devices include infusion sets, pump reservoirs, syringes, pens, vials and cartridges.

### CROSS-REFERENCE TO RELATED APPLICATION

This application claims priority to U.S. Ser. No. 61/810,111, filed Apr. 4, 2013, to Gerard Michel, Robert Feldstein, Robert Hauser, and Bryan R. Wilson, the disclosure of which is incorporated herein by reference.

### FIELD OF THE INVENTION

This invention is in the field of devices and methods for mixing components to form pharmaceutical formulations immediately prior to use.

### BACKGROUND OF THE INVENTION

Many pharmaceutically active components, notably proteins and peptides, are not stable in aqueous solution or suspension at room temperature, for prolonged storage prior to use in a form that provides optimal pharmacokinetic (PK) and pharmacodynamic (PD) profiles. Modifying such formulations to improve their shelf-life typically modifies their pharmacokinetic and/or pharmacodynamics profiles. Therefore unstable pharmaceutically active agents are often mixed with excipients, additional active agents, and/or diluents to form the desired drug formulation immediately prior to administering the formulation to a patient.

For example, with respect to the administration of insulin to a patient, it is difficult to preformulate insulin with EDTA and citric acid due to the impact that the excipients (EDTA and citric acid) have on the stability of the resulting formulation.

Similarly, the use of glucagon to manage moderate hypoglycemia and hyperinsulinemia is difficult because glucagon rapidly degrades after reconstitution, and variable doses are required to treat these indications. Currently glucagon is reconstituted immediately prior to use, and the required amount of the formulation is used, with any remaining formulation discarded due to its instability. Thus much of the formulation is wasted.

Additional steps are currently required prior to administering various formulations, which increases the complexity and costs associated with the administration of the formulation. For example, the preinfusion of magnesium sulfate to improve the tolerability of injectable formulations with poor tolerability, such as propofol, requires an additional step prior to administration of the drug formulation compared to the administration of the drug in the absence of magnesium sulfate. The preinfusion of hyaluronidase to enable subcutaneous administration of intravenous drugs similarly requires an extra step. Further, sometimes coformulation is difficult or impossible due to compatibility issues between the drug and hyaluronidase,

The recommended period of use for an insulin pump infusion site is three days. The preinfusion of hyaluronidase to improve the consistency of insulin absorption over the course of the three day treatment requires an additional step. The need for this additional mixing step immediately prior to use may limit the usefulness of the formulation. Coformulation is difficult due to compatibility issues between insulin and hyaluronidase. Additionally, the insulin formulation and hyaluronidase are generally manufactured and distributed by different companies.

A common form of point of use mixing is reconstituting drugs just prior to use. The glucagon emergency kit is used for intervention in hypoglycemia. The glucagon solution is prepared for injection immediately before use. A vial of diluent is provided in a pre-filled syringe, is added to dry glucagon powder in a separate vessel, and is shaken until the glucagon dissolves and the solution becomes clear. The same syringe is then used to withdraw the solution (glucagon plus diluent) from the vessel and into the syringe. Then the solution is injected into the patient via subcutaneous injection. Any unused material is discarded due to the instability of reconstituted glucagon.

Other point of use mixing systems include dual bore syringes, which can deliver combined, simultaneous liquid dosing, and dual chamber pumps, which can be used to provide simultaneous outputs, although this is not their normal mode of operation.

However, each of the known systems has its challenges, including multiple steps and/or vials, delays associated with the mixing, and/or incomplete mixing, as well as wasted drug after mixing due to instability of the mixed formulation. These challenges can result in slower or faster drug release, administration of lower or higher than needed amounts of the drug, and/or higher costs. Some of these challenges can produce devastating results. For example, when administering a dose of insulin required to treat diabetes, a small difference in the dose can result in the difference between good gylcemic control and hypoglycemia.

Therefore, there is a need for improved methods and devices for administering unstable compounds, which provide acceptable storage stability without increasing the complexity of the administration.

There is a need for improved devices and methods that efficiently and completely mix drug formulations immediately prior to use, do so on-demand, and/or for variable amounts of drug formulation.

There is a need for improved methods for storing and administering drug formulations.

It is an object of the invention to provide improved devices and methods for mixing drug formulations.

It is a further object of the invention to provide systems for storing and mixing drug formulations prior to administration.

### SUMMARY OF THE INVENTION

Methods and devices for delivering variable amounts of a drug formulation directly from a drug delivery device without requiring point of use reconstitution as a separate mixing step are described herein. These methods and devices increase the convenience, speed and simplicity of administration of drug formulations. The devices mix two or more components in a fixed volume ratio effectively simultaneous to administration, and allow for variable doses while retaining the desired ratio of the components in each dose. The mixing devices are particularly useful for administering final formulations that are unstable at room temperature in liquid form.

Although the disclosure focuses on pharmaceutical applications, the devices described herein may be used in other settings, such as chemicals, foods, cosmetics, and general industrial environments where a fixed ratio of two or more components is desired in of point of use mixing.

The mixing devices can be incorporated into or attached to existing drug delivery devices, such as pump reservoirs, infusion pump sets, pens (e.g. insulin pens), or syringes. In one embodiment, excipients are mixed into a drug formulation at a fixed excipient to drug formulation volume ratio as the drug formulation flows from the cartridge, reservoir or syringe barrel into the patient. For example, the excipients may be added in a pump infusion set so that they are mixed with the drug formulation as the drug formulation is flowing through the infusion set to the user. In another embodiment, such as for a pen (e.g. NovoPen®, KwikPen®) used in multiple daily injections (MDI), needle hubs are described which can be used with disposable pen needles to add the excipients during the actual injection of the drug formulations. In another embodiment, the cartridges and vials add the excipients as the drug formulation is transferred from the cartridge or vial.

In some embodiments, the excipients are contained within a reservoir in the device, either fixed or flexible; in others the excipients are loaded within a porous solid, such as an open cell foam, sintered metal beads, or mesh.

In all of the embodiments, at least one component of the two or more components that are mixed to form the final drug formulation is in a liquid form. Optionally, both components are in a liquid form. In some embodiments the excipients are provided in a liquid form, e.g. as a solution or a suspension. In other embodiments, the excipients are provided in a dry solid or in the form of a powder. In some embodiments, the drug is provided in a dry form. In other embodiments the drug is provided in a liquid form, e.g. as a solution or a suspension.

### BRIEF DESCRIPTION OF THE DRAWINGS

FIG. 1 is a schematic of an insulin infusion set used to add a liquid excipient or diluent to the flow path of a solution or suspension containing the active agent, just prior to the point of administration to the patient.

FIG. 2 is a perspective view of a needle hub reservoir for a liquid/liquid mixing system.

FIGS. 3A and 3B are schematics of another liquid/liquid mixing system with a linear arrangement of the reservoir and conduit. FIG. 3A is a perspective view, with the internal components visible. FIG. 3B is a cross-sectional view.

FIGS. 4A-C are schematics of a further liquid/liquid mixing system with the reservoir and conduit aligned coaxially. FIG. 4A is a plan view. FIG. 4B is a cross-sectional view taken about line A-A of FIG. 4A. FIG. 4C is a three-dimensional view of the cross-section depicted in FIG. 4B.

FIGS. 5A and 5B are views a needle hub containing a solid/liquid mixing system. FIG. 5A is a plan view of the needle hub. FIG. 5B is a cross-sectional view taken about line A-A of FIG. 5A.

FIGS. 6A and 6B are views of a needle hub containing different solid/liquid mixing systems. FIG. 6A is a plan view of the needle hub. FIG. 6B is a cross-sectional view taken about line A-A of FIG. 5A.

FIG. 7 is a perspective view of a needle hub reservoir containing solid excipient particles between an inlet needle and an outlet needle, so that liquid drug mixes with the excipient as it passes between one needle to the next.

FIG. 8 is a graph of mean baseline subtracted insulin concentration uU/ml) as a function of time (minutes) in diabetic miniature swine following administration of 0.25 U/kg of BIOD-403 (solid line) or Humalog® (dashed line), n=9, ±SEM.

FIG. 9 is a graph of mean baseline subtracted glucose values (mg/dL) as a function of time (minutes) for two different formulations, BIOD-403 (solid line) and Humalog® (dashed line) in miniature diabetic swine, n=9, ±SEM.

FIG. 10 is a schematic of the primary components in a mixing system that includes a collapsible bag, where the bag is impermeable to the fluid inside the bag and the fluid outside of the bag. The bag contains one or more excipients that can mix in a desired ratio at the desired time with a drug, just prior to administration of the final drug formulation.

FIGS. 11A-C are different views of an exemplary vial containing a co-axial liquid-liquid mixing device (depicted in FIGS. 4A-C) with a moveable barrier. FIG. 11A is a side view; FIG. 11B is a cross-sectional view taken about line A-A on FIG. 11A; and FIG. 11C is an magnified view of portion “B” on FIG. 11B.

FIG. 12A-C are different views of an exemplary cartridge containing a co-axial liquid-liquid mixing device (depicted in FIGS. 4A-C) with a moveable barrier. FIG. 12A is a side view; FIG. 12B is a cross-sectional view taken about line A-A on FIG. 12A; and FIG. 12C is an magnified view of portion “B” on FIG. 12B.

### DETAILED DESCRIPTION OF THE INVENTION

Methods and devices for delivering variable amounts of a drug formulation directly from a drug delivery device without requiring point of use reconstitution as a separate mixing step are described herein. These methods and devices increase the convenience, speed and simplicity of administration of drug formulations. The devices mix two or more components in a fixed volume ratio effectively simultaneous to administration, and allow for variable doses while retaining the desired ratio of the components in each dose. The devices are particularly useful for administering drug formulations that are unstable at room temperature in liquid form.

The methods described herein generally use conventional devices that have been modified to administer an unstable compound and provide acceptable storage stability. Typical devices include syringes and pens. The devices described herein mix two components in a precise mixing ratio immediately prior to administration, and allow for variable doses. In preferred embodiments, the variable doses retain a constant molar ratio of the components in each dose.

The methods and devices allow one to deliver a drug that is unstable in aqueous solutions, such as glucagon, directly from a syringe or pen device, without requiring point of use reconstitution as a separate mixing step. These methods and devices increase the convenience, speed and simplicity of administration of drug formulations. The devices may be used to treat a variety of diseases and disorders, including severe hypoglycemia.

In some embodiments, the device mixes two liquid components to form the formulation for administration to a patient. In other embodiments, the device mixes a liquid component and a dry component to form the formulation for administration to a patient. Each of the liquid or dry components contains drug, one or more excipients and/or a solvent. Following mixing the resulting formulation contains a combination of drug, solvent and/or one or more excipients in a precise ratio regardless of the dose chosen. Thus the devices can administer variable doses with the ratio(s) of drug, excipient(s) and/or solvent remaining the same without introducing any extra steps to the user.

The mixing devices generally contain a flow diversion barrier which directs a portion of liquid of the first component (based on the desired mixing ratio) to bypass the second component and allows for release of a desired amount of the liquid of the first component to mix with the second component to form the final drug formulation. In the liquid-liquid mixing embodiments, the mixing device also contains a second barrier that prevents the entire volume of liquid of the first component that was not bypassed from mixing with and diluting the second component and forces the same volume of the second liquid component liquid to mix with the volume of the bypassed first component liquid to form the final drug formulation. Examples of this second barrier include but are not limited to movable barriers, collapsible barriers, and open cell porous solids.

A variety of different configurations for the mixing device may be used. Preferably the mixing device is inexpensive to manufacture and easy to use. The mixing device may be attached to the drug delivery device such that it is removable therefrom, or it may be integrated into the drug delivery device.

### I. DEFINITIONS

The terms “drug” and “active agent” are used synonymously herein and refer to chemical or biological molecules providing a therapeutic, diagnostic, or prophylactic effect in vivo.

As used herein “excipients” refer to any ingredients included in a pharmaceutical formulation for the purpose of giving a suitable consistency, form, or properties, such as release properties and/or pharmacokinetic and/or pharmacodynamic profiles, to a drug.

As used herein an “initial drug formulation” refers to a drug formulation prior to mixing with additional excipients. Often the initial drug formulation contains one or more excipients prior to mixing. The drug that is mixed in the devices described herein may be included in an initial drug formulation.

As used herein “final drug formulation” refers to the drug formulation that is administered to a patient. For example, the final drug formulation results from the mixture of the desired amount of excipients with a drug or an initial drug formulation.

As used herein “reservoir” in a mixing device refers to a portion of the mixing device that stores a component (liquid or solid form).

As used herein “conduit” in a mixing device refers to a portion of the mixing device through which a fluid can flow.

As used herein “mixing ratio” refers to both volumetric mixing ratios and molar ratios of the materials that mix to form the final drug formulation. The volumetric mixing ratio refers to the mixing that occurs between two liquid components as a portion (a given volume) of a liquid exits a reservoir and mixes with a second liquid (of a second volume) that was in a second reservoir or a conduit. The term molar ratio is generally used to refer to the molar ratio of a drug to one or more added excipients or a first drug to a second drug in a final drug formulation.

### II. DEVICES FOR LIQUID/LIQUID POINT OF USE MIXING

Liquid diluents, optionally containing one or more excipients, may be added to and mixed with liquid solutions or suspensions containing an active agent. In all of the embodiments described herein, the final drug formulations have a constant volumetric ratio of initial drug formulation component to liquid diluent or liquid excipient component. Further in those embodiments where the mixing device does not contain a one-way valve (either virtual or mechanical), the final drug formulations have a constant molar ratio of drug to excipient.

A. Mixing Devices with Bypass with One-Way Valves or Open Cell Foam

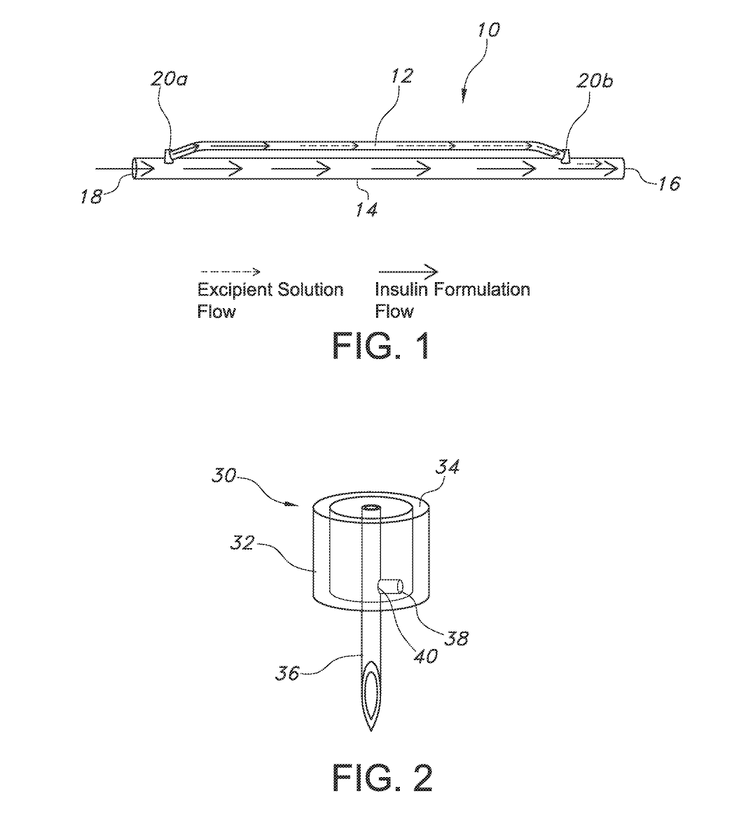

In one embodiment the mixing device contains a one-way valve. An exemplary device is illustrated in FIG. 1. The mixing device may be a component in an infusion set 10 and used to add a liquid diluent, optionally containing one or more excipients, to a liquid drug formulation. The device contains a primary infusion line 14 and a secondary infusion line 12. The primary and secondary lines are connected to each other at both an inlet end 22 and a second outlet end 24, which attaches to the infusion site in a patient. Resistances or flow barriers 20a and 20b are located at each end where the secondary line attaches to the primary line.

The inlet end 22 is in fluid communication with a pump or pump reservoir 18 (not shown in figure).

In use, a formulation containing an active agent, preferably in a higher concentration than will be administered to the patient, is pumped into the inlet end 22 of the primary infusion line 14. A flow resistance is the primary path that provides the pressure drop required to divert a portion of the flow in the primary infusion line 14 into the secondary infusion line 12. This displaces a portion of the excipient, which will mix with the primary flow downstream of the resistance, which controls the mixing ratio. A flow barrier in the excipient infusion line (i.e. the secondary infusion line 12) prevents mixing between the diverted active agent (e.g. insulin) and the excipient. The barrier can have any suitable shape and be formed from any suitable material. The diluent or excipient solution is released into the formulation containing an active agent through the resistance 20b at the outlet end. This allows the formulation to mix with the excipients just prior to administration to the patient.

a. One-Way Valve

In some embodiments, the flow restrictions are one-way valves. One way valves prevent the diluent or excipient solution or suspension in the secondary line 12 from entering the primary infusion line 14 at the pump end, thereby minimizing residence time of the combined formulation within the primary infusion line 14.

In some embodiments, the barrier is a small close fitting sphere.

b. Open Cell Foam

In an alternative embodiment, the resistance may be formed by an open cell porous solid, i.e. a foam, located at each end where the secondary line attaches to the primary line. In place of an open cell foam, other porous solids, such as sintered metal beads, or a mesh, may be used.

When a fluid is in contact with a wet able surface, the contact angle between the fluid and the surface is characteristic of the “wet ability” of the surface, and is generally thought of as the contact between a “surface film” and the adjacent phase, the so called “surface tension”. For pores with dimensions comparable to the radius of the surface contact, i.e. small pores, the surface tension serves to confine the liquid within the pore, mechanically. This requires force to displace the liquid (i.e., overcome the surface wetting adhesion) and is usually represented by an average over the interface as “pore pressure”. The result is that liquid contained within a network of pores tends to be captive, and requires pressure to displace it. In the absence of pressure, a foam barrier between liquids tends to act as a barrier against the inter diffusion between the liquids, since mixing occurs only by pore to pore diffusion, which is a slow process that gets progressively less efficient the deeper into the foam, where the concentration gradient gets smaller and smaller.

An applied pressure produces a flow through the foam, which while symmetric, will act only in the direction of force. If the pressure is unidirectional the flow will be unidirectional, e.g. mimicking the flow through a one-way valve (described above).

Preferably the open cell foam is a hydrophilic small-cell foam with a controlled porosity, such as one with a similar hydrophilicity to a polyurethane foam. In the preferred embodiment the foam is a polyurethane open-celled foam. In contrast, foams without well-defined, controlled pore size, distribution and structure, such as a cellulose foams, are not generally useful for the devices disclosed herein is.

A limitation of a one-way valve system, mechanical or a “virtual” one-way valve, such as created with an open cell foam, is that the fluid in the reservoir becomes progressively diluted by the addition of the fluid introduced to displace the reservoir contents for mixing. Progressive dilution occurs over the use cycle. Therefore, the reservoir is preferably large and/or highly concentrated to limit the dilution to an acceptable degree over the use cycle. Additionally, the reservoir should contain additional excipient as a safety factor.

Alternatively, the mixing device can have a suitable configuration for use in a disposable or reusable device, such as a syringe or pen. A mixing device 30 that can be attached to or integrated into a syringe or pen is depicted in FIG. 2. This mixing device 30 contains a needle hub 32 for use with syringe or pen device. The needle hub 32 contains a reservoir 34, which is configured to contain an excipient solution or suspension. The reservoir 34 is connected to the needle 36 via a port 38 containing a flow resistance element 40, such as a one-way valve or an open cell foam.

a. One-Way Valve

In use, the valve closes when the formulation containing an active agent is being drawn into the syringe (not shown, connects to upper portion of needle hub 30). The driving force is suction. When a patient self-administers a formulation by injection, the patient inserts the needle into the site of administration and depresses a syringe plunger in the device. The pressure from depressing the syringe plunger opens the valve 40, which draws the excipient solution or suspension into the stream of the formulation containing the active agent. In this manner the active agent mixes with the excipients and/or diluent immediately prior to injection into the patient.

b. Open Cell Foam

Optionally, the device contains an open cell foam in the same location within the device as the one-way valve (i.e., element 40). The open cell foam is described above.

B. Mixing Devices with Moveable Barriers

Alternatively, the mixing device contains a moveable barrier. These devices are preferred, particularly for use in relatively small (e.g. hand-held) devices, such as a pen, since the mixing of the excipients with the initial drug formulation does not dilute the excipients. Thus the composition containing the excipients need not be as concentrated as it should be in the devices or systems utilizing a one-way valve.

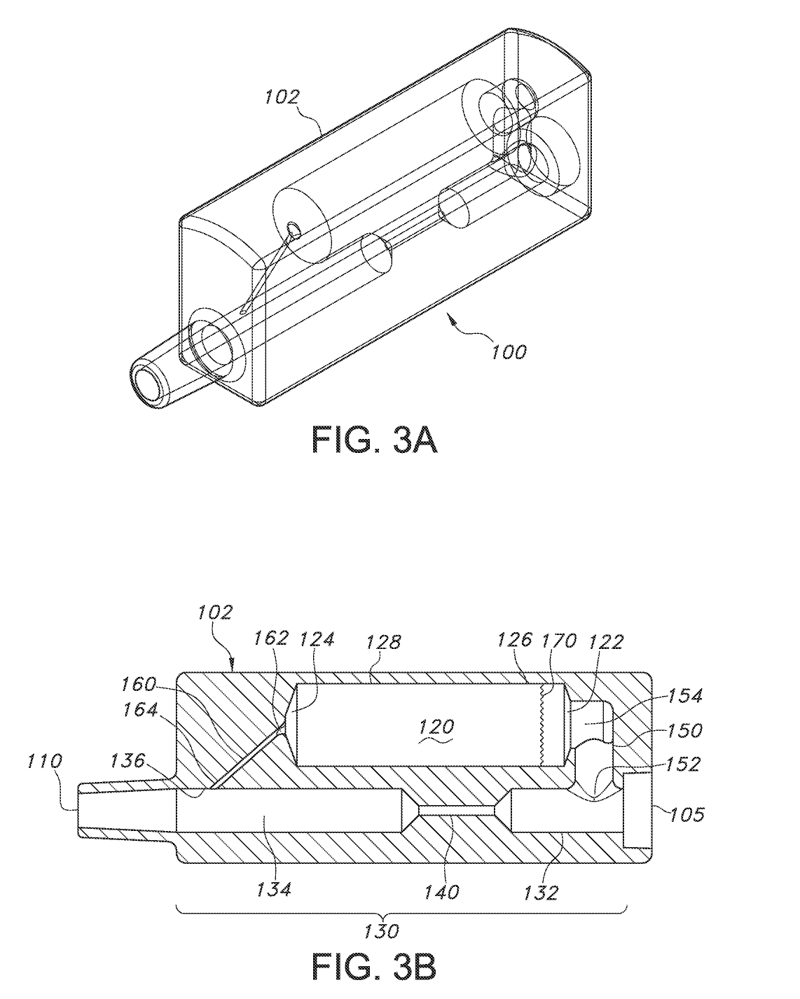

Exemplary mixing devices that utilize a moveable barrier are depicted in FIGS. 3A-B and 4A-C. These devices can be used with an infusion device, such as a reservoir pump and infusion set; an injectable device, such as a syringe or pen; or a component of an injection pen, such as a cartridge, or a vial for a syringe. These devices for mixing liquid precursor solutions to form a final drug formulation typically contain a reservoir and a conduit, which attaches to the inlet and the outlet for the device. The conduit contains a restriction area (R1), which has a smaller diameter than the rest of the conduit. The reservoir connects to the conduit via a second restriction (R2), which typically has a smaller diameter than the diameter of the restriction area in the conduit. The excipient in the liquid precursor solution is typically at a high concentration to allow a large mixing ratio and minimize the dilution caused by the addition of the excipient.

Optionally a free floating barrier or membrane may be included in the inlet side of the reservoir to prevent diffusion mixing of the inlet and reservoir fluids. Since both fluids are incompressible, no force is applied to the barrier or membrane. The barrier or membrane may be formed of any material that maintains its shape and orientation, without inhibiting motion. Suitable barriers or membranes include but are not limited to a molded ring, a screen or film ring, or an O-ring of any insoluble material. Suitable materials include but are not limited to Teflon, silicon rubber, polyethylene, and stainless steel.

a. Linear Arrangement of Reservoir and Conduit

As shown in FIGS. 3A and 3B, in one embodiment, the mixing device 100, contains an inlet 105, an outlet 110, a reservoir 120, and a conduit 130. As illustrated in the cross-sectional view shown in FIG. 3B, the reservoir and conduit are arranged along essentially parallel lines, referred to herein as a “linear arrangement”. The reservoir and conduit are inside a device housing 102.

The conduit 130 contains an inlet portion 132 and an outlet portion 134, which are connected to each other by a first restriction (referred to herein as “R1”) 140. The reservoir contains a first opening 122 located at its inlet side 126 and a second opening 124 located at its outlet side 128. Optionally the reservoir contains a free floating barrier or membrane 170 in the inlet side 126 of the reservoir to prevent diffusion mixing of the inlet fluid and the fluid in the reservoir. The barrier or membrane may be formed or any material that maintains its shape and orientation, without inhibiting motion. Suitable barriers or membranes include but are not limited to a molded ring, a screen or film ring or an O-ring of any insoluble material. Suitable materials include but are not limited to Teflon, silicon rubber, polyethylene, and stainless steel.

In some embodiments, a membrane or barrier is not present in the reservoir. In these embodiments some mixing will occur between the liquid component that enters the device through the inlet 105 and the liquid component stored in the reservoir 120. This device may be particularly useful when an excipient (i.e. the component in the reservoir) is very concentrated and only a small portion is used before the unit is discarded. Then excipient concentration will not vary unacceptably.

The inlet portion of the conduit is in fluid communication with the reservoir via a channel 150. The channel 150 contains two openings on its opposite ends. The channel connects via the first opening 152 with the inlet portion 132 of the conduit, and connects via the second opening 154 with the first opening 122 of the reservoir.

The reservoir is in fluid communication with the outlet portion of the conduit via a second restriction (referred to herein as “R2”) 160. The second restriction contains two openings, one at each of it opposite ends. The first opening 162 of the second restriction connects with the second opening 124 of the reservoir. The second opening 164 of the second restriction connects directly with an opening 136 into the outlet portion 134 of the conduit.

The inlet portion 132 of the conduit is in fluid communication with the inlet 105 of the mixing device.

The outlet portion 134 of the conduit is in fluid communication with the outlet 110 of the mixing device.

The inlet 132 and outlet portions 134 have little resistance compared to R1 or R2. Therefore the ratio of R2 to R1 controls the mixing ratio of the component in the reservoir (e.g. one or more excipients) to the active agent component (which enters the device through the inlet 105).

b. Coaxial Arrangement of Reservoir and Conduit

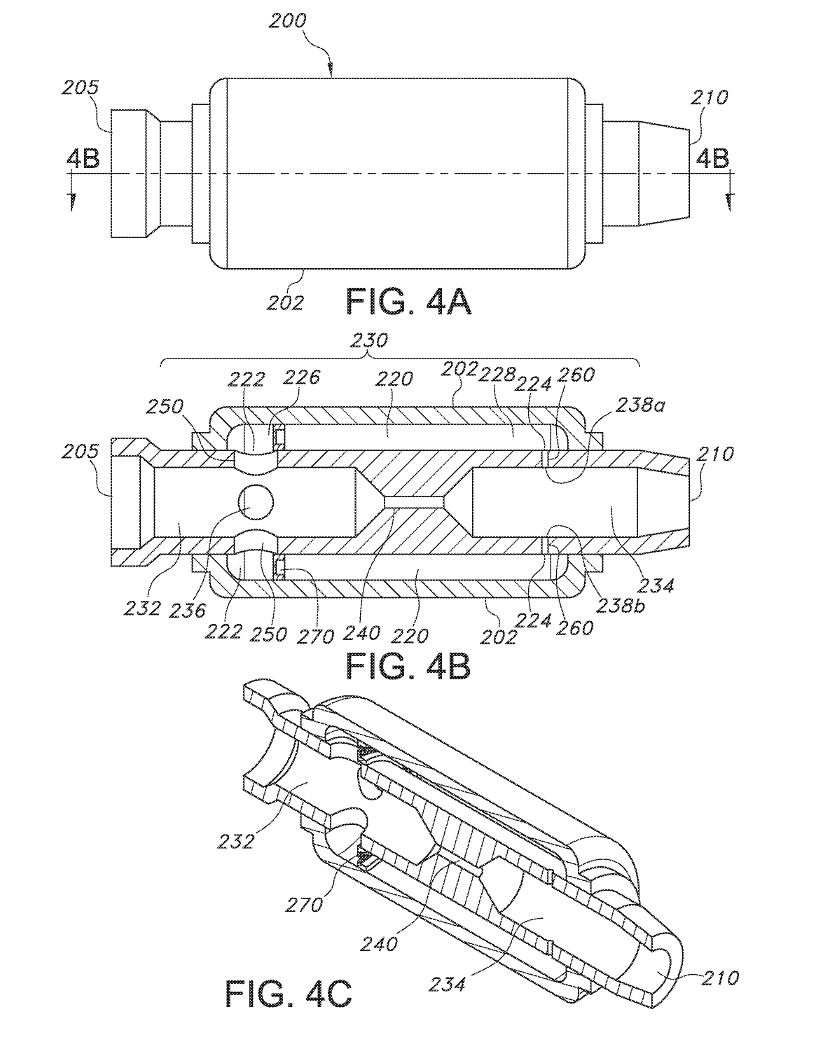

As shown in FIGS. 4A-C, the mixing device 200 contains an inlet 205, an outlet 210, a reservoir 220, and a conduit 230. The reservoir and conduit are inside a device housing 202. As illustrated in the cross-sectional view shown in FIG. 4B, the reservoir is parallel and annularly arranged relative to the conduit, referred to herein as a “coaxial arrangement”. This arrangement is preferred relative to a linear arrangement, particularly for use with a pen or syringe, since it allows for reduction in the overall size of the device.

The conduit 230 contains an inlet portion 232 and an outlet portion 234, which are connected to each other by a first restriction (abbreviated herein as “R1”) 240.

The reservoir contains a first opening 222 located at its inlet side 226 and a second opening 224 located at its outlet side 228. Optionally the reservoir contains a free floating barrier or membrane 270 initially in the inlet side 226 of the reservoir 220 to prevent diffusion mixing of the fluid component in the inlet of the conduit and the fluid in the reservoir. The volume of the reservoir is typically very small.

The inlet portion 232 of the conduit is in fluid communication with the reservoir 220 via a channel 250. This is a low resistance channel 250 connecting the inlet portion 232, i.e. before R1 240 (moving from the inlet 205 to the outlet 210), to the reservoir 220 at essentially equal pressure. The inlet portion 232 contains one or more relatively large openings 236 in fluid communication with the channel 250.

The reservoir 220 is in fluid communication with the outlet portion 234 of the conduit via a second restriction (abbreviated herein as “R2”) 260. The outlet portion 234 contains two small openings 238a and 238b that connect with the second restriction (R2) 260 to allow the fluid component in the reservoir to enter into the outlet portion and mix with the fluid component in the conduit, prior to exiting the outlet of the device and administration to the patient.

As described above with respect to the device depicted in FIGS. 3A and 3B, the ratio of the diameters for R1 240 and R2 260 controls the mixing ratio of the components.

The inlet portion 232 of the conduit is in fluid communication with the inlet 205 of the mixing device.

The outlet portion 234 of the conduit is in fluid communication with the outlet 210 of the mixing device.

The sizes for the conduit, reservoir and restrictions are determined by the desired mixing ratio for each specific drug/excipient combination, the volume of drug being used, and the amount of excipient (and/or solvent) needed. Once the diameters are selected, the length of the conduit sets the available volume of drug, with no effect on mixing ratio. This allows multiple uses of the system without significant change. The length of the reservoir is also determined by the volume of excipient required.

For example, for an insulin pass-through, if the concentration of the excipient in the reservoir is at 10 times the concentration of the total excipient in the final formulation, then the volume of the excipient component that is added to the fluid in the outlet portion of the conduit is about 0.1 ml per 100 IU. Thus the reservoir is very small. The flow resistance is concentrated at the exit of the excipient reservoir and opening 238a and 238b to the outlet portion 234 of the conduit. The entire excipient reservoir is at hydrostatic equilibrium with the inlet pressure. As the excipient component enters the outlet portion of the conduit, the free floating barrier or membrane 270 moves along the reservoir, from the inlet side 226 of the reservoir 220 toward the outlet side 228.

C. Mixing Devices with a Collapsible Barrier

In some embodiments, the mixing devices contain a flexible, collapsible barrier that is impermeable to both of the liquids in the device. For example, the collapsible barrier is preferable in the form of an expandable and collapsible bag. The impermeable, flexible barrier separates the inlet and reservoir fluids to prevent diffusion mixing of the inlet and reservoir fluids. Since both fluids are incompressible, the inlet fluid pushes or flexes the barrier wall and forces excipient out of the reservoir. Suitable barrier materials include but are not limited to Teflon, silicon rubber, and polyethylene.

The collapsible barrier is preferably in the form of a bag and is attached to a portion of the device at the opening of the bag, such that it is stationary at the attachment (e.g. has stationary edges).

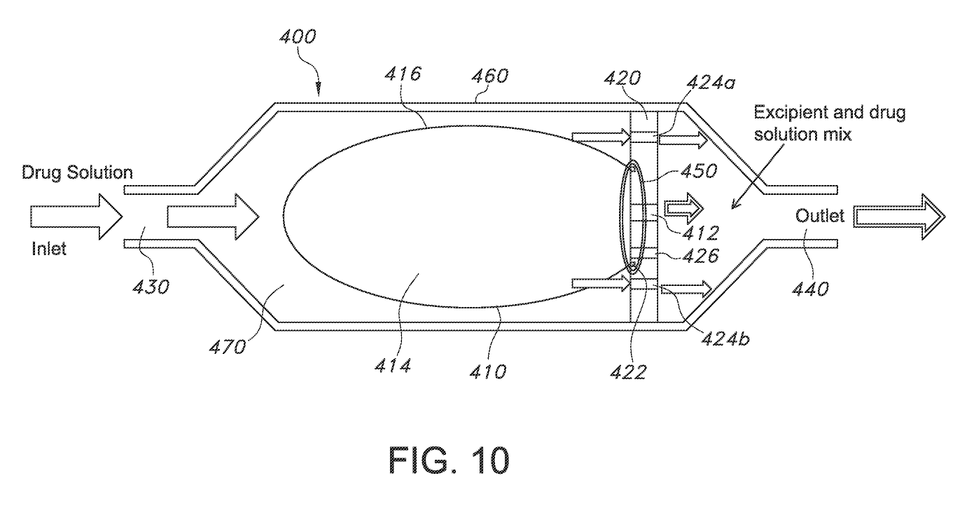

FIG. 10 depicts an exemplary mixing device with a flexible, collapsible, impermeable barrier. As shown in FIG. 10, the mixing device 400 contains flexible, collapsible barrier 410 which is in the form of a bag. The opening 412 of the bag is configured to receive and release excipient; and the inside 414 of the bag is configured to store excipient, i.e. serves as the excipient reservoir. The device contains an opening at each end, were one opening serves as an inlet 430 and the other serves as an outlet 440, where the inlet and outlet are in fluid communication with each other. An attachment portion 420 is located inside the device, between the inlet and outlet. The edges of the opening to the bag are attached to the attachment portion by suitable connecting means, such as by fitting into a groove or channel 422 in the attachment portion, optionally held in place with an O-ring 450 or other suitable fixation means (e.g. adhesive, friction fit). Inside the groove or channel 422 is an opening 426 for the transfer of excipient fluid into or out of the bag. The attachment portion also includes one or more restrictions 424a and 424b to control the transfer of the component containing the drug (e.g. an initial drug formulation) from the inlet to the outlet and to create more pressure on the outer wall 416 of the bag. The restrictions are narrow channels in the attachment portion.

The attachment portion also contains a gas vent 426, which is in fluid communication with the inside of the bag. The gas vent is a small diameter opening that allows gas out of the bag when the bag fills with excipient, but is sufficiently small not to allow leakage of fluid from the bag.

The mixing device 400 can be assembled by attaching the opening 412 of the bag to the attachment portion, e.g. inserting it into the groove and fixing it in place with an integral terminal O-ring. The attachment portion, with its attached bag is inserted into device housing 460, e.g. a cylinder, preferably oriented vertically, with the outlet end up, positioned and secured in place. Preferably end caps (not shown in figure) are placed on each opening and secured in place.

The bag can then be filled with the desired quantity of the appropriate liquid, e.g. a fluid component containing the excipient, using a standard method. For example, the bag can be filled by orienting the mixing device vertically, with the outlet end up, and injecting the fill through a needle placed against the shoulder of the center hole recessed chamfer formed by the outlet end countersink (needle stop). The needle is captive to the cylindrical outlet end extension of the center hole, and can be guided into position by a final (exit surface) counter sink, in case there is an alignment error in needle placement. The inner set of small hole adjacent to the center hole are sufficiently small to provide an acceptably small additional contribution to the center hole effective radius but will provide and effective gas venting system, during bag fill, due to the low viscosity of gas and the available driving pressure provided by an incompressible liquid injected via a needle with an effective shoulder seat, which is provided by the needle mechanical down force on the stop.

The inlet and outlet geometry can be modified as desired, to permit integration of a Lauer lock, hypodermic needle, crimp seal, etc. The mixing device may be integrated into a pump, vial, cartridge, pen, hypodermic syringe, or another two component mixing device or system.

In use, the mixing device operates by a feed liquid stream that is pressure driven through a flow resistance (see, e.g. 424a and 424b in FIG. 10). The upstream pressure is exerted on a liquid reservoir by the incompressible liquid in the conduit (470) surrounding the reservoir (e.g., 414), and the liquids in the reservoir and conduit are separated from each other by the collapsible, impermeable barrier (410), with minimum pressure drop. The second liquid is mixed with the initial stream through a second controlled flow resistance (orifice). The resistance ratio is adjusted to produce a controlled, reproducible mixing ratio between the two liquids, prior to the outlet.

D. Mixing Devices with Open Cell Porous Solid

In some embodiments, the mixing devices contain an open cell porous solid, such as an open cell foam, sintered beads or mesh loaded with the excipients. The same porous solid materials as described above may be used in these embodiments.

This open cell porous solid is in effect a floating barrier with no moving parts. The open cell porous solid creates a “virtual” barrier, which is preferred compared to one-way valves and moveable barriers due to its simplicity and lack of dilution during use.

For example, in one embodiment an entire reservoir is filled with an open cell porous solid, such as a hydrophilic foam, with all the pores initially filled with one of the required fluids. The reservoir need only be large enough to hold the required fluid volume, plus the extra volume occupied by the foam and a safety factor of excess fluid (e.g. excess diluent/excipient or drug to ensure there is a sufficient amount).

The incompressible fluid (e.g. diluent/excipient component or drug containing component) will be captive to the foam, by pore pressure, and will not diffuse away in storage, or significantly evaporate, since the adjacent volume, if any, will saturate. Once pressure is applied to one end of the foam filled reservoir fluid will be displaced, and a fluid interface will progress through the reservoir. The excess pressure required to produce the displacement is added to the total fluid resistance in that flow path, and can be compensated for by altering concentration and/or altering the resistance of the flow path. The interface between the reservoir fluid and the driving fluid will broaden with time, via diffusion. However, if the cell size is sufficiently small, the diffuse layer thickness will not reach the outlet end of the reservoir prior to the end of use. The exit fluid will remain undiluted, due to the “virtual” barrier maintaining a separation between the driving and exiting fluids.

In some embodiments, the excipient component is captured in the porous solid. In other embodiments, the drug component is captured on the porous solid. The liquids are incompressible; and vapor pressure at the exposed ends of the porous solid competes with pore pressure to limit evaporation. Pore pressure limits diffusion as excipient (or drug) is displaced by carrier solution, maintaining a consistent molarity (of the captured component) at the exit end of the porous solid.

1. Methods of Using Liquid/Liquid Point of Mixing Devices

A concentrated additive material reservoir is prefilled with a liquid, which typically contains one or more excipients to be combined with the solution introduced at the inlet. The channel connecting the inlet to the reservoir is of relatively large diameter and offers essentially no flow resistance. Therefore the inlet pressure will be uniformly distributed to the inlet side of R1 and the entire reservoir up to R2, since liquids are incompressible, for the mixing devices depicted in FIGS. 3B and 4B.

The relative flow rates for the fluid out of the reservoir and into the conduit and the fluid flowing through the conduit (from the inlet portion to the outlet portion) are inversely proportional to the size of the restrictions, R1 and R2. If R1 is a larger bore, i.e. has a larger diameter, than R2, then R1 provides a lower resistance to fluid flow. Therefore a larger volume of fluid will flow through R1 than R2 in a given time period. The diameter and length of each restriction is selected to provide the desired mixing profile, i.e. the flux and driving pressures each fluid moving through the mixing device.

The “mixing ratio” is the ratio of the volume of excipient fluid to the volume of “flow through” fluid at the outlet of the mixing system. The mixing ratio is determined by the ratio of the diameter of R1 to the diameter of R2, if the following conditions are met. First the flow velocity must be low enough to be far from the onset of turbulence, and second the viscosity of the two fluids is comparable. The first condition is met when the mixing device is attached to or integrated in a standard drug delivery device or system, such as a syringe, pen, or pump, and flow profiles with stationary fluid at the wall (surface) is an accurate approximation. The viscosity of the two fluids may not be the same, particularly if the reservoir contains an ultra-concentrated liquid, i.e. excipients in a higher concentration, such as ten times or greater than 10 times than the concentration of the excipient(s) that will be present in the resulting drug formulation. However, the difference in viscosities for a particular set of fluids may be compensated for by adjusting bore length or diameter in one of the resistances.

Additionally, the fluid ejected from the reservoir is replaced by fluid from the inlet, which represents a loss of inlet fluid. Therefore an ultra-concentrated reservoir fluid, allowing a very high mixing ratio (inlet to reservoir fluid), provides a minimum loss of incoming fluid (which serves as a hydraulic “driver”). This reduces the amount of dilution of the fluid in the conduit, such that there is no material impact on the drug concentration and thus no need to modify the drug concentration in the component provided to the device through the inlet.

The flow resistance is concentrated at the exit of the excipient reservoir and openings to the outlet portion of the conduit. The entire excipient reservoir is at hydrostatic equilibrium with the inlet pressure. As the excipient component enters the outlet portion of the conduit, some of the fluid in the inlet component enters the reservoir pushing the free floating barrier or membrane 270 such that it moves along the reservoir, from the inlet side 226 of the reservoir 220 toward the outlet side 228.

The diameters and lengths for R1 and R2 are preferably selected to achieve the desired mixing ratio for a given set of components (e.g. one or more excipients and/or solvent in one component and drug, optionally with solvent, in the second component). In some embodiments, typical diameters for the restrictions range from 10 to 50 thousandths of an inch. In some embodiments, typical lengths for the restrictions range from ⅛ inch to ¼inch.

The fluids mix in the outlet portion of the conduit to form a final drug formulation, and the drug formulation exits the mixing device through the outlet. The mixing device is attached to a suitable drug delivery device. Thus as the final drug formulation exits the mixing device, it is administered to a patient, either via injection using a syringe or pen, or as an infusion.

### III. MIXING DEVICES FOR LIQUID/SOLID POINT OF USE MIXING

A two-component system, which contains excipients in the dry form and drug in a liquid form or drug in a dry form and diluent, optionally with one or more excipients (in a liquid form) are described herein. The mixing devices described herein produce final drug formulations that have a constant molar ratio of drug to excipient.

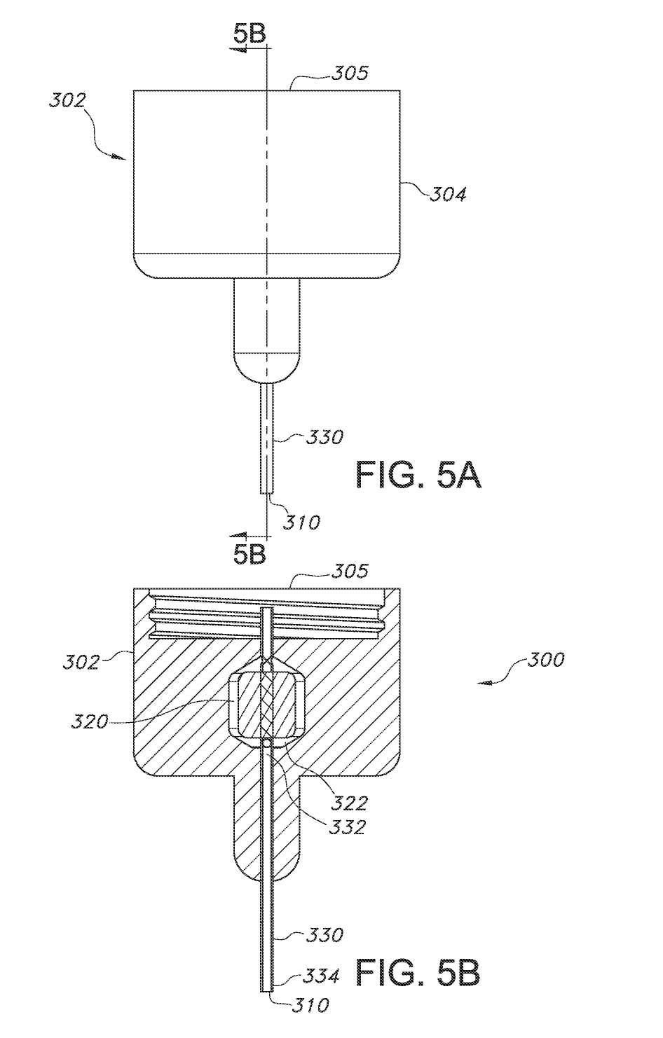

FIGS. 5A-B and 6A-B are illustrations of exemplary liquid/solid point of use mixing devices, for a solid (pellet) or powder, dry system. Alternatively, these mixing devices can be used with a porous solid, such as an open cell foam, sintered metal beads, or mesh.

Porous Solid with Dried Excipients or Drug

The porous solid is typically soaked with the excipient or drug formulation to be captured in the solid and then freeze dried (e.g. lyophilized) to form a dry porous solid with the drug or excipients. The porous solid provides a relatively large surface area in a relatively small volume and can be used in any drug delivery device, In use, for example if excipients are dried in and on the porous solid, then as a drug formulation moves through the porous solid, it becomes saturated with the excipients in a known mixing ratio. In addition to use with syringes or pens, such as in the devices depicted in FIGS. 5A-B and 6A-B, the porous solid with dry drug or excipients can be used in other delivery devices and systems, such as infusion set, pump reservoir, or related components, such as in a cartridge or vial.

A. Solid for Storing Drug

The dry form of most drugs is more stable and can be stored for a longer time period than the liquid form.

These devices can be used to store a drug, such as glucagon, in a dry form and add a diluent, optionally with one or more excipients, from a reservoir (syringe, pen auto injector or other liquid source) just prior to injection. The diluent dissolves the dry material. Then the combined solution exits via the needle to the subcutaneous injection depot.

B. Solid for Storing Excipients

Alternatively, these devices can be used with more stable drugs, such as drugs that are stable for at least one year at 5° C. in liquid form. In these embodiments, the solid may contain one or more excipients. The use of a solid, rather than a liquid, to add the excipients allows for a more compact additive. This allows for a more compact mixing device than would be needed if a liquid additive was used.

For example, the excipients could be ones that modify the pharmacokinetic (PK) profile of the drug. For example, a liquid stable drug formulation like Humalog® (Insulin lispro, Eli Lilly & Co.) can be modified at the point of administration by combining the liquid formulation with excipients, such as ethylenediaminetetraacetic acid (EDTA) and trisodim citrate, that modify its PK profile.

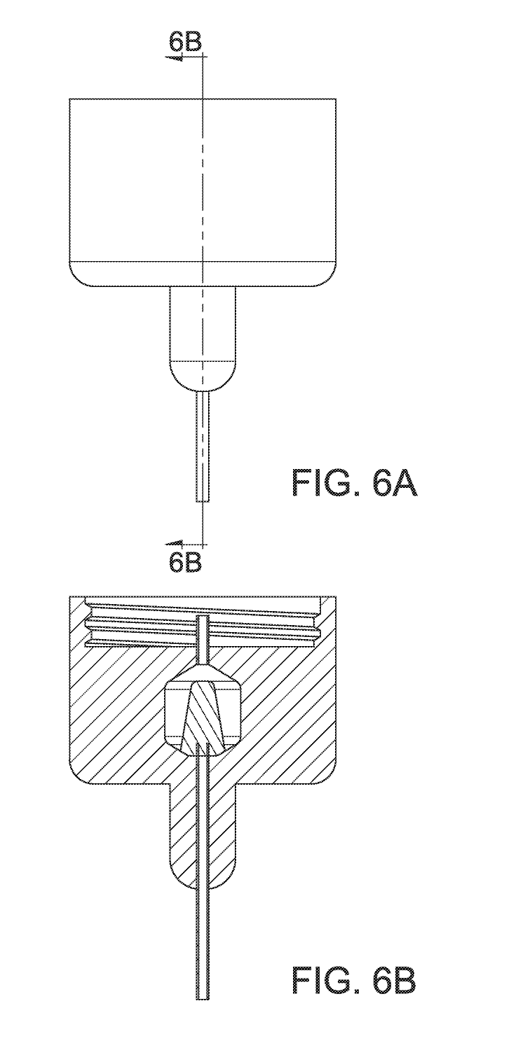

As illustrated in FIGS. 5B and 6B, the mixing system can contain a compressed tablet or a porous solid containing one or more excipients or the drug in dry form. Preferably the compressed tablets contain a binder. These devices are preferably single-use, disposable devices.

The mixing device 300 contains a needle hub 302, which includes a housing 304 and a needle 330, and a chamber 320 with a sufficient size and shape to contain a solid, compressed tablet. Typically, the needle hub is prepackaged and contains the solid compressed tablet.

The chamber is inside the housing and surrounds the needle. Optionally the walls of the chamber are porous. The walls may be laser drilled to provide the desired porosity.

The chamber 320 is a well in the hub and prior to use contains a seal or diaphragm at its proximal end, which maintains the solid material in a sealed environment for storage. The diaphragm or seal must be peeled or punctured at the time of use.

The needle optionally contains a rear pointing section (proximal end) 332 to puncture the seal or diaphragm) covering at least a portion of the proximal end 322 of the chamber 320 until it is to be used. Immediately prior to use, the patient applies pressure to the needle, such that the proximal end of the needle punctures the separator, providing access to the solid material in the chamber.

The needle hub also contains an inlet 305 and an outlet 310. The outlet 310 is located at the distal end 334 of the needle 330.

The chamber has a suitable size and shape to fit inside a standard needle hub. The chamber has a smaller diameter and length than the diameter and length of a standard needle hub.

In one embodiment, illustrated in FIG. 6B, the walls 322 of the chamber 320 are porous. The walls of the chamber may be laser drilled 5 or 6a can be made this way

The solid in the chamber may be in the form of a tablet. Alternatively, it may be in the form of a coated foam or porous structure. The solid in the chamber may be a powder, where the grain size of the powder may be controlled to control the rate of dissolution of the solid.

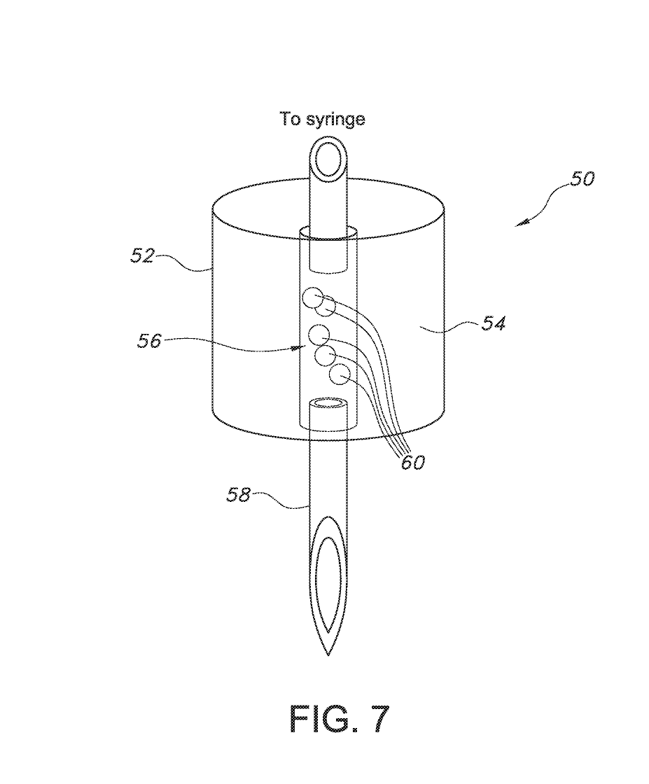

A simplified needle hub mixing device is illustrated in FIG. 7. In this embodiment, the mixing device 50 is also configured in the needle hub 52 for use with a syringe or pen, preferably the resulting drug formulation is an insulin or insulin analog formulation. The needle hub 52 contains a reservoir 54 containing the excipient as a solid 56. The excipient reservoir 54 is connected to the needle 58 via ports 60. The ports may be laser drilled holes that are small enough to act as zero pressure valves, i.e. at low pressure surface tension will prevent fluid flow.

In use, the valve closes when the solution is being drawn into the syringe, and the dry material partially dissolves. The driving force is suction. The pressure from depressing the syringe plunger forces the liquid back through the remaining powder, causing the drug solution to dissolve the dry excipient 56 prior to the administration to the patient.

C. Methods of Using Liquid/Solid Point of Mixing Devices

The concentration of the combined solution exiting the mixing device can vary from near saturation initially (assuming the flow velocity is low enough to allow the solid and liquid to come to equilibrium) to diluent at the end of the injection. Preferably excess diluent is provided to ensure virtually complete dissolution of the dry material. Thus, in a single injection, the concentration of the drug in the drug formulation varies. However, all of the drug is administered over the course of a single injection.

There is a possibility that un-dissolved fragments of the solid material can be entrained in the exit stream and plug the exit bore. Optionally, the mixing device contains a screen or other size barrier to prevent plugging the exit bore (needle for injection) with small particles. Small particles that pass through the screen or barrier, then pass through the exit bore (i.e. needle for injection), later dissolving in the extra cellular fluid in the depot in the patient's subcutaneous skin at the exit of the system.

The solid component may contain either the excipient or the drug.

The mixing ratio should be controlled to ensure that complete mixing occurs to form a drug formulation with the desired PK or PD profile. The mixing can be controlled by the solubility of the solid system and the portion of the liquid that flows through, rather than around, the solid reservoir. Although the mixing achieved in the solid/liquid mixing devices may be less accurate than the liquid/liquid systems described above, such a liquid/solid mixing device is generally appropriate for applications in which the mixing ratio is relatively non critical. For example, this mixing device is useful for single use, fixed dose injections. These systems depend only on delivering the entire active ingredient and are independent of the co-delivery of an inactive component. This delivery system may be used to mix and deliver vaccine antibodies, glucagon, and vitamin mega doses. For glucagon formulations, the amount of glucagon delivered matters; however the amount of liquid in which it is delivered does not have a significant effect. The liquid generally is an inactive agent, such as saline or water.

The mixing devices described herein are generally described in use with devices for injection to deliver injectable formulations, however the advantages of the concept of point of use mixing apply to devices for other routes of administration, such as for the delivery of dosage forms suitable for nasal or oral administration.

For example, an inhaler or nasal spray device may contain a reservoir immediately prior to the exit point to facilitate mixing of the pharmaceutically active components in the formulation. These devices can be modified in a variety of manners. For example, the devices may contain two reservoirs that connect to each other and are in fluid communication with a common exit port, mixing on contact and/or on the impact surface.

Coformulation

Although the devices are generally described with respect to mixing a drug with a diluent and/or excipients immediately prior to or simultaneous with administering the final drug formulation to a patient, these systems and devices can be used to mix two, and optionally more than two, different initial drug formulations. The mixing devices can be used to co-formulate two or more drug formulations and to co-administer two (or more) drugs. This is preferably used with drugs that cannot co-reside in storage. Optionally, the mixing devices contain an additional reservoir containing a second drug or second drug formulation or a buffer.

The devices described herein are intended to provide illustrations of possible ways of implementing the mixing methods and are not meant to be limiting. Many additional variations and alternatives will be apparent to those of ordinary skill in the art.

### IV. USES FOR THE MIXING DEVICES

The methods and mixing devices described herein can be used to prepare and deliver a variety of different formulations, which are otherwise too unstable to administer to a patient without the use of additional mixing steps prior to administration.

For example, excipients that improve the pharmacokinetics of insulin or insulin analogs, such as EDTA and citric acid, may be mixed as described herein to provide an insulin formulation with the desired PK profile.

Magnesium sulfate may be added to injectable formulations immediately prior to administration using one or more of the methods and devices described herein to improve tolerability of injections with poor tolerability (such as propofol).

Hyaluronidase can be coadministered with an infusion using one or more of the devices and methods described herein to enable subcutaneous administration of intravenous drugs.

Hyaluronidase can be coadministered with an insulin pump to improve the consistency of insulin absorption during the period of use (typically three days) for an insulin pump infusion site.

Glucagon can be administered via injection to a patient using one or more of the mixing devices described herein to manage mild to moderate hypoglycemia and hyperinsulinemia (an orphan disease). In one embodiment, a highly concentrated nonaqueous solution (which is stable but not suitable for chronic administration) is mixed at a fixed ratio with an aqueous diluent immediately prior to administration. This allows for a multi-dose, room temperature stable product for chronic administration of variable doses. Some of these uses are described in more detail below.

A. Incorporation into or Attachment to Existing Drug Delivery Devices

The mixing devices can be incorporated into or attached to existing drug delivery devices, such as infusion pump sets, pens (e.g. insulin pens), or syringes. In an alternative embodiment, the excipients are added as the drug formulation is transferred from a vial into a syringe or other delivery device. In one embodiment, excipients are mixed into a drug formulation at a fixed excipient to drug formulation ratio as the drug formulation is flowing from the cartridge, reservoir or syringe barrel into the patient. For example, the excipients may be added in a pump infusion set so that they are mixed with the drug formulation at a fixed ratio as the drug formulation is flowing through the infusion set to the user. In another embodiment, such as for a pen (e.g. NovoPen®, KwikPen®) used in multiple daily injections (MDI), needle hubs are described which can be used with disposable pen needles to add the excipients during the actual injection of the drug formulations. In a further embodiment, disposable, single use syringes that contain a mixture of excipients which are separated from the drug to be delivered are described. These syringes are useful for vial using MDI patients.

The devices have the excipient and mixing mechanisms integrated into each of the devices (needle, syringe, infusion set) so the user has no additional steps to contend with when setting up or using the pen, insulin syringe or infusion set. The devices allow the user to bolus or inject a variable amount of insulin units as they would with the existing devices.

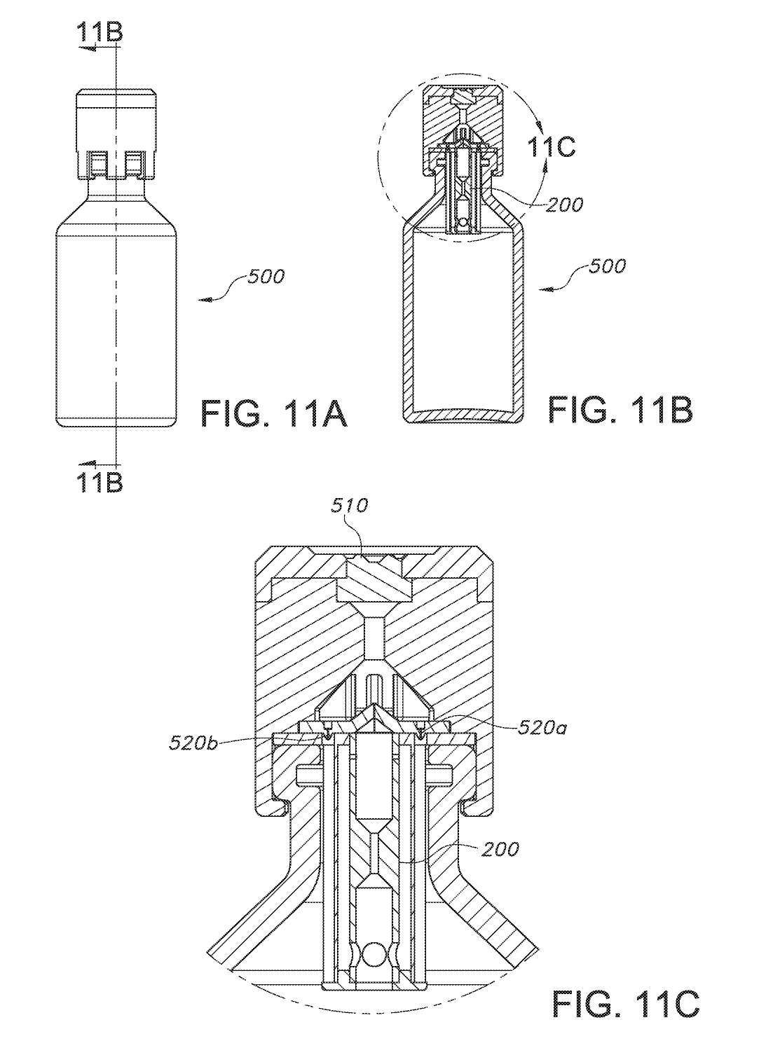

An exemplary vial containing a liquid/liquid mixing device, such as depicted in FIGS. 4A-C is provided in FIGS. 11A-C. The vial 500 includes the mixing device 200 in the neck of the vial. The vial is configured to allow a user to pressurize the vial by inserting a needle of a syringe into and through the rubber septum 510, the air flows into the vial, thereby creating positive pressure to enable the removal of the formulation from the vial, through the mixing device 200 and into the syringe.

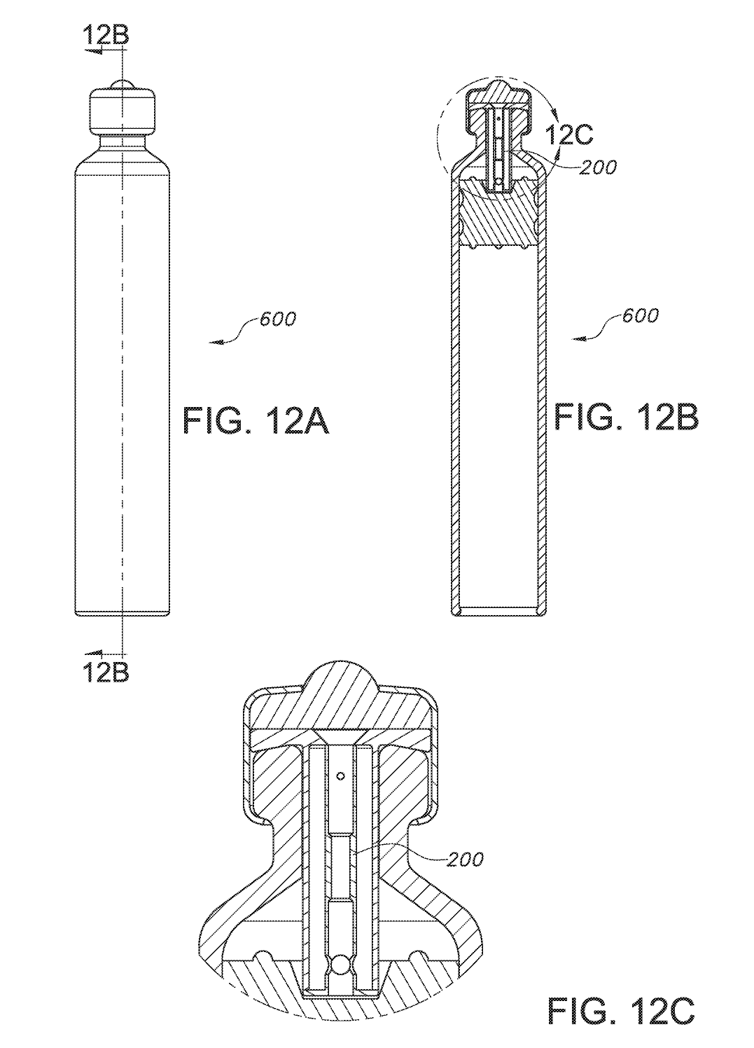

An exemplary cartridge 600 containing a liquid/liquid mixing device 200 is provided in FIGS. 12A-C. The cartridge is configured to fit in a standard pen device for administration of a final drug formulation.

B. Modification of PK/PD Profiles for Insulin Analogs

Rapid-acting insulin analogs, such as Humalog®, Novolog® and Apidra®, have faster absorption than recombinant human insulin (RHI) since they have different amino acid sequences which produce a “looser” hexamer that more readily dissociates upon injection. With an RHI based formulation there is a slightly slower off (or “tail”) than existing analogs and with an analog based formulation there is a slightly faster off (or shorter tail) than RHI.

Some multiple daily injection (MDI) patients believe that the current analogs are too fast off, while some others want a faster off. In reality it is highly situation dependent (meal content, activities pre and post meal) as well as patient dependent. For pump patients, there is general consensus that the ability to easily take a correction bolus that is both “fast on” and “fast off” would be ideal.

The addition of excipients to RHI or an insulin analog leads to a formulation with a PK/PD profile that is significantly “faster on” than the original formulation.

The study described below demonstrates that point of use mixing can be used to produce formulations with a desired pharmacokinetic profile, such as an ultra-rapid acting insulin or insulin analog (URAI) formulation.

URAI formulations developed by Biodel, such as described in U.S. Published Application No. 2010/0227795A1 to Steiner, et al., and U.S. Published Application No. 2012/0178675A1 to Pohl, et al. (the disclosures of which are incorporated herein by reference), have faster rates of absorption than both regular human insulin and rapid-acting insulin analogs, and pharmacokinetic and pharmacodynamic (PK/PD) profiles which are closer to the first phase insulin response profiles seen in individuals not afflicted with diabetes. Biodel's formulations are based on the use of excipients (EDTA, citrate and, optionally, magnesium sulfate) which create monomeric, charge masked insulin which is rapidly absorbed upon injection. Because only a very small amount of Biodel's proprietary excipient is required to improve the PK profile of RHI, aspart, lispro, or glulisine, the excipients can be added automatically to the currently commercially available products via a specially designed insulin needle, insulin syringe or infusion set. Additionally, it is believed that the ratio of excipient to insulin (or insulin analog) formulation can vary significantly (+/−30% or more) and still produce a desirable “faster on” PK profile.

The mixing devices described herein can be used to improve the PK profile of Apidra®, Humalog®, and/or Novolog®. They can also be used to improve the PK profile of RHI such that its speed of onset is faster than that of Humalog® and Novolog®. While the specific mixing location and mechanics vary for each device, the effect is the same; a final insulin formulation that has an URAI profile. These formulations can be used by patients with type 1 and type 2 diabetes mellitus to control their glucose levels.

C. Glucagon Formulations

Pharmacologically, glucagon increases the concentration of glucose in the blood. Following injection, the immediate pharmacologic result is an increase in blood glucose at the expense of stored hepatic glycogen. The onset of action post injection is 5-20 minutes. Glucagon is degraded in the liver, kidney, and tissue receptor sites. The half-life of glucagon in plasma is 3 to 6 minutes, which is similar to that of insulin.

Glucagon is unstable in aqueous solution at room temperature.

Currently, the commercial preparation of glucagon is a two part sterile vial, intended for immediate use following reconstitution. It is sold as a rescue kit and is available for intravenous, intramuscular or subcutaneous administration. The kit contains 1 mg (1 unit) of glucagon and 49 mg of lactose in a sterile vial. The diluent contains 12 mg/mL glycerin, water for injection and hydrochloric acid. The diluent is injected into the powder vial and gently swirled to dissolve the glucagon; then the glucagon solution is pulled back into the same syringe ready for injection. The pH of this solution is approximately 2. The recommended dose is typically 0.5-1 mg. Any reconstituted glucagon is to be discarded since it is not stable in solution.

The devices described herein can be used to mix glucagon formulations immediately prior to use. For example, 1 mg of glucagon in dry form (with a stabilizer) may be mixed with a diluent, such as sterile water at pH2 (added HCl) plus any additives, such as glycerin.

### Example

### Mixing of Liquid Excipients with Humalog® Immediately Prior to Injection to Create an Ultra-Rapid-Acting Insulin Formulation

### Feasibility Study in Diabetic Swine

This study was conducted to evaluate in diabetic swine the pharmacokinetics of URAI formulations when Humalog® is mixed with an excipient mixture just prior to injection. The excipient mixture was a 20× concentrated excipient mixture containing EDTA and sodium citrate.

Methods and Materials

The composition of the excipients in the 20× solution was 48 mg/ml trisodium citrate, 9 mg/ml disodium EDTA and 9.62 mg/ml MgSO₄.

To simulate the effect of mixing just prior to injection, 0.05 ml of 20× concentrated liquid excipients was drawn into a 1 cc syringe. Then 30 seconds prior to administration, 0.95 ml of commercially supplied Humalog® was drawn into the same syringe, mixing with the excipients as the syringe was filled. Then, the appropriate dose was administered to each swine on a weight basis (0.25 U/kg).

The control formulation was mixed identically, with the exception that saline was used in place of the excipient mixture.

Using a crossover study design, nine fasted diabetic miniature swine were intravenously cannulated and placed in a sling the morning of the study.

Immediately after mixing, the swine were injected with the initial insulin formulation plus excipient solution (BIOD-403) at a dose of 0.25 U/kg. Humalog® controls were prepared with saline in the same manner. Immediately post dose, swine are fed 500 g of swine diet. Plasma samples were collected at −30, −20, −10, 0, 5, 10, 15, 20, 30, 45, 60, 75, 90, 120, 150, 180, 240, 300, 360 min post dose and blood samples were placed directly into a Becton Dickinson K₂EDTA vacutainer tube. Plasma was collected and frozen until assayed for insulin concentration (Iso-insulin Kit, Mercodia) and glucose concentration (YSI 3200 analyzer, YSI Life sciences, USA).

Results

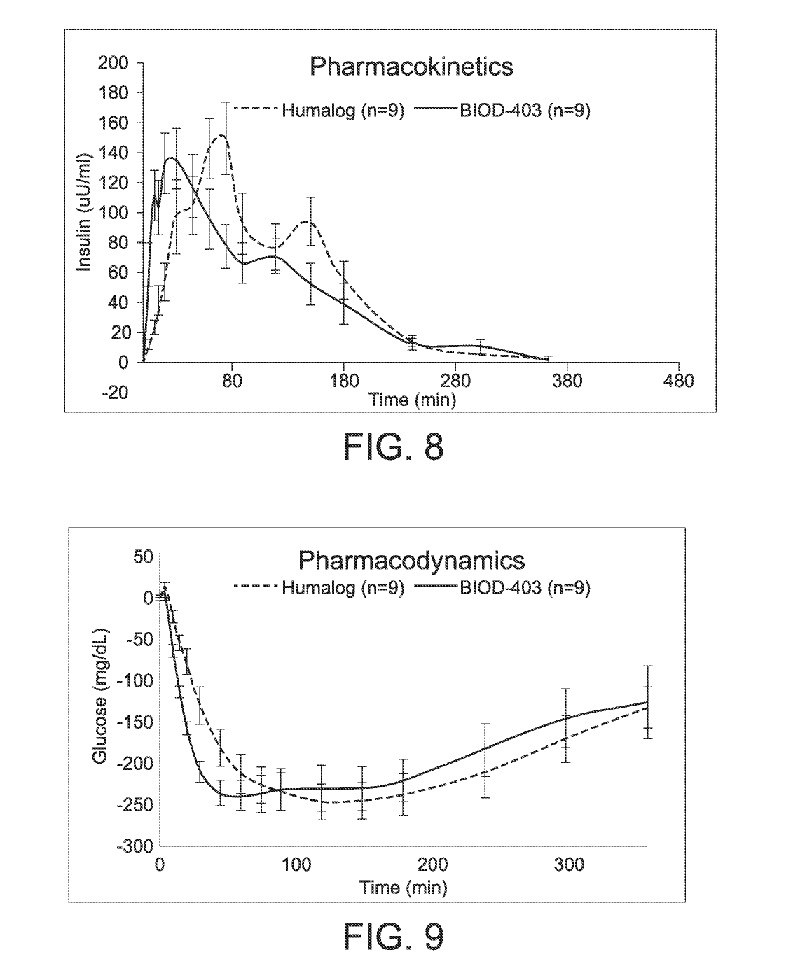

The pharmacokinetic profiles of these formulations are shown below in FIGS. 8 and 9 and pharmacokinetic parameters are provided in Table 1.

TABLE 1

| | | | P |
|---|---|---|---|
| | Humalog | BIOD-403 | value |
| Cmax | 182 ± 20 | 166 ± 17 | p = .55 |
| (uU/mL) | | | |
| Tmax (min) | 70 ± 12 | 20 ± 2 | p = .0008 |
| T_{1/2 max} (min) | 31 ± 4 | 8.8 ± 2 | p = .0002 |
| AUC | 18918 ± 1925 | 16881 ± 2125 | p = .25 |
| (uU/mL * min) | | | |

### CONCLUSIONS

The mixing of concentrated liquid excipients with Humalog® just prior to administration statistically reduces the time to Cmax; and the pharmacokinetic profile is similar to formulations made up of the same components in a pre-mixed form.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

1. A method for forming a dose of an injectable final drug formulation immediately prior to or at the time of administration to a patient comprising:
combining in a mixing device two liquid components to form the final drug formulation,
wherein at least one of the components comprises a drug to be delivered,
wherein the mixing device comprises a reservoir and a conduit,
wherein a volume of liquid comprising the first component passes through the conduit to mix with a volume of liquid comprising the second component that exits the reservoir to form the final drug formulation, and
wherein the volume of liquid exiting the reservoir is a function of the volume of liquid passing through the conduit such that in the final drug formulation the ratio of the liquid from the reservoir to the liquid that passed through the conduit is constant regardless of the dose administered to the patient.

2. A method for forming a dose of an injectable final drug formulation immediately prior to or at the time of administration to a patient comprising:
combining in a mixing device a solid component and a liquid component to form the final drug formulation,
wherein at least one of the components comprises a drug to be delivered,
wherein the mixing device comprises a first reservoir and either a second reservoir or a conduit,
wherein a volume of liquid exits the second reservoir or conduit and mixes with the solid component to form the final drug formulation, and
wherein the mixing ratio first component and the second component is constant regardless of the dose administered to the patient.

3. The method of claim 1, wherein the final drug formulation is unstable at room temperature.

4. The method of claim 1, wherein the first component comprises the drug and the second component comprises one or more excipients, and wherein the molar ratio of the drug to the excipients is constant regardless of the dose administered to the patient.

5. The method of claim 1, wherein the mixing device is a part of a delivery device or system that administers the final drug formulation to the patient.

6. The method of claim 5, wherein no additional steps are required to administer the final drug formulation to the patient.

7. The method of claim 1, wherein the mixing device is in a vial or a cartridge.

8. The method of claim 1, wherein the mixing device further comprises a flow diversion barrier, wherein the diversion barrier directs a fixed ratio of liquid in the conduit to bypass the conduit and allows for release of a desired amount of the liquid from the reservoir to mix with the first component in the conduit to form the final drug formulation.

9. The method of claim 8,
wherein the mixing device comprises a second barrier, wherein the second barrier prevents the entire volume of liquid of the first component that was not bypassed from mixing with and diluting the second component.

10. The method of claim 9, wherein the second barrier is selected from the group consisting of movable barriers, collapsible barriers, and open cell porous solids.

11. The method of claim 8, wherein the flow diversion barrier is one or more restrictions in a conduit, preferably two restrictions, wherein the first restriction (R1) and the second restriction (R2) have different diameters.

12. The method of claim 1, wherein the first component comprises the drug and the second component comprises a solvent, one or more excipients, or a combination thereof.

13. The method of claim 1, wherein the first component comprises the drug and the second component comprises a second drug.

14. The method of claim 1, further comprising a third component.

15. The method of claim 1, wherein one component consists of an initial drug formulation comprising insulin or an insulin analog and wherein the second component comprises excipients that are effective to modify the pharmacokinetics of the initial drug formulation to produce the final drug formulation with the pharmacokinetics of ultra-rapid acting insulin or insulin analog (URAI) formulation.

16. A mixing device for preparing a final drug formulation from two liquid components, or a liquid and a solid component, wherein one of the components comprises a drug to be delivered and the second component comprises a solvent, one or more excipients, or a combination thereof wherein at least one of the components comprises a drug to be delivered,
wherein the mixing device comprises a reservoir, a conduit, and a barrier, wherein the barrier is configured allow for release of a desired amount of the liquid in the reservoir, such that the volume of liquid exiting the reservoir is a function of the volume of liquid passing through the conduit, wherein in the final drug formulation, the ratio of the liquid from the reservoir to the liquid that passed through the conduit is constant regardless of the dose administered to the patient.

17. The mixing device of claim 16, wherein the barrier is selected from the group consisting of movable barriers, collapsible barriers, and open cell porous solids.

18. The mixing device of claim 16 in a second device selected from the group consisting of infusion sets, pump reservoirs, disposable syringes, and a disposable needle.

19. The mixing device of claim 16 in a vial or pen cartridge.

20. The mixing device of claim 16, wherein the barrier is a movable barrier,
wherein the conduit comprises an inlet portion, an outlet portion and a first restriction, wherein the first restriction has a narrower diameter than the diameter of either of the inlet and outlet portions and wherein the first restriction (R1) connects the inlet portion to the outlet portion,
wherein the reservoir comprises an inlet end and an outlet end,
wherein the conduit and reservoir are in fluid communication with each other, and wherein the inlet portion of the conduit is connected to an opening in the inlet end of the reservoir by a channel, and
wherein the device further comprise a second restriction (R2), wherein the second restriction connects the outlet end of the reservoir to the outlet portion of the conduit, and wherein the second restriction has a different diameter than the diameter of the first restriction.

21. The mixing device of claim 18, wherein the barrier is a free floating barrier located in the inlet side of the reservoir to prevent diffusion mixing of the fluid in the inlet portion of the conduit and the fluid in the reservoir.

22. The mixing device of claim 18, wherein the ratio of the diameters for the first and second restrictions ranges from 10:1 to 1:10.

23. The mixing device of claim 16, wherein the reservoir comprises an excipient solution or suspension, wherein when mixed with the drug, the excipient modifies the PK/PD profile of the drug in the final drug formulation.

24. The method of claim 1, further comprising administering the final drug formulation via an infusion set, pump reservoir, drug pen, or syringe.

25-30. (canceled)

31. The method of claim 1, wherein the reservoir is surrounded by a collapsible, impermeable barrier, and wherein the liquid in the conduit exerts a pressure on the liquid in the reservoir, wherein the liquid exits the reservoir through a controlled flow resistance and mixes with the liquid in the conduit.

32. The mixing device of claim 16, wherein the barrier is a collapsible, impermeable barrier, and wherein the reservoir is surrounded by the barrier, wherein the reservoir further comprises a controlled flow resistance configured to release a controlled volume of the liquid from the reservoir and allow it to mix with the liquid in the conduit, and wherein the conduit is configured such that the liquid in the conduit exerts a pressure on the liquid in the reservoir.

33. The mixing device of claim 16, wherein the device is configured to fit inside the outlet of a pen cartridge or vial for a syringe.
